(19) United States
(12) Patent Application Publication
(10) Pub. No.: US 2009/0233327 A1
(43) Pub. Date: Sep. 17, 2009

(54) Surface Modification in a Manipulation Chamber

(75) Inventors: Aldrich N.K. Lau, Palo Alto, CA (US); Huan L. Phan, Belmont, CA (US)

Correspondence Address: LIFE TECHNOLOGIES CORPORATION;C/O INTELLEVATE, P.O. BOX 52050, MINNEAPOLIS, MN, 55402, US

(73) Assignee: Life Technologies Corporation, Carlsbad, CA (US)

(21) Appl. No.: 12/398,140

(22) Filed: Mar. 4, 2009

Related U.S. Application Data
(63) Continuation of application No. 11/423,810, filed on Jun. 13, 2006, now abandoned.
(60) Provisional application No. 60/731,123, filed on Oct. 27, 2005.

(51) Int. Cl. C12Q 1/02; B05D 5/12; B05D 3/00

(52) U.S. Cl. 435/29; 427/58; 427/108; 427/457; 427/384; 427/398.1; 427/331

(57) ABSTRACT

A device for manipulating biological material, the device including at least one electrode and a photoconductive material configured to receive the biological material; and a light source configured to illuminate the photoconductive material so as to modulate an electric field, wherein the electric field is configured to manipulate the biological material; wherein a surface of the at least one electrode and/or the photoconductive material is modified with at least one of a carboxylic moiety, an amino moiety, a poly(ethylene glycol) moiety, a polymer of (poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), and a poly((meth)acrylamide) derivative. Methods for manipulating biological material are also disclosed.

### RELATED APPLICATION

This application is a continuation of U.S. application Ser. No. 11/423,810 filed Jun. 13, 2006, which claims the benefit of U.S. Provisional Application No. 60/731,123 filed on Oct. 27, 2005, which are incorporated herein by reference.

### TECHNICAL FIELD

The present teachings relate to methods and devices for manipulating biological material such as, for example, nucleic acids, proteins, enzymes, cells, biological particles, and other micro-particles and/or nano-particles.

### BACKGROUND

Cellular analysis and research often requires the manipulation of small particles, such as biological material, including nucleic acids, proteins, enzymes, cells, cell aggregates, cell organelles, stem cells, bacteria, protozoans, viruses, and/or other micro- and/or nano-particles. Typically, the small particles to be manipulated have a dimension (e.g., diameter) ranging from approximately 0.1 micrometer to approximately several hundred micrometers, for example from approximately 1 micrometer to approximately 100 micrometers, or, for example, from approximately 5 micrometers to approximately 10 micrometers. By way of example only, mammalian cells have a diameter ranging from about 5 micrometers to about 100 micrometers and a lymphocyte can be about 10 micrometers in diameter. In some cases, groups of particles (e.g., cells, stem cells, etc.) can be separated from other particles. The dimension of a group of particles can be as large as about 100 micrometers.

Various devices and methods have been used to manipulate small particles so as to identify, discriminate, sort, characterize, quantitate, observe, move, collect, and/or otherwise manipulate the small particles, such as, for example, live stem cells. For example, a technique dubbed “optical tweezers” has been developed which uses a high intensity laser to manipulate and trap micron sized objects in a surrounding medium, such as an aqueous suspension. Using optical tweezers, the laser beam induces optical gradient fields, which generates a radiation pressure force that can capture and manipulate micrometer-scale particles in the aqueous suspension. Exemplary applications utilizing the principles of optical tweezers is discussed in, for example, Ashkin et al., “Optical trapping and manipulation of single cells using infrared laser beams,” Nature, vol. 330, December 1987, pages 769-771; and Arai et al, “Tying a molecular knot with optical tweezers,” Nature, vol. 399, June 1999, pages 446-448, each of which is incorporated by reference herein.

Other techniques for manipulating small particles include the use of dielectrophoretic force. Dielectrophoresis (DEP) refers to the motion imparted on uncharged objects as a result of polarization induced by a spatially non-uniform electric field. An analytical expression of the dielectrophoretic force, \vec{F}_{DEP}, acting on a particle (T. B. Jones, Electromechanics of Particles, Cambridge University Press, 1995) is set forth below:

$${\vec{F}}_{DEP} = 2 π {r}^{3} {ɛ}_{m} {α}_{r} \vec{∇} ( {\vec{E}}_{RMS}^{2} ) , \\ {α}_{r} ≡ Re ( \frac{{ɛ}_{p}^{*} - {ɛ}_{m}^{*}}{{ɛ}_{p}^{*} + 2 {ɛ}_{m}^{*}} )$$

In the above equation, r is the radius of the particle, the factor in parentheses in the first line of the equation is the RMS value of the electric field, and αᵣ is the real part of the Clausius-Mosotti factor which relates the complex permittivity of the object ∈ₚ and the complex permittivity of the medium ∈ₘ. The star (*) denotes that the complex permittivity is a complex quantity. The Clausius-Mosotti factor can have any value from 1 to −½, depending on the AC frequency used to generate the electric field, and the complex permittivities of the object and the medium. If it is less than zero, the dielectric force is negative and the particle moves toward a lower electric field. If the Clausius-Mosotti factor is greater than zero, the dielectric force is positive and the particle moves toward a stronger electric field.

If the particles are charged, then under DC current or low frequency AC current, electrophoresis (EP) occurs, instead of DEP. EP refers to the lateral motion imparted on charged objects in a non-uniform or uniform electric field.

DEP has been used to manipulate particles, such as cells, for example, via a traveling wave generated by a series of patterned electrodes lining up and charged with phase-shifted AC signals. The electrodes can be patterned in an independently controlled array to provide the traveling wave. For examples of such a technique, reference is made to Pethig et al., “Development of biofactory-on-a-chip technology using exciter laser micromachining,” Journal of Micromechanics and Microengineering, vol. 8, pp. 57-63, 1998, and Green et al., “Separation of submicrometer particles using a combination of dielectrophoretic and electrohydrodynamic forces,” Journal of Physics D: Applied Physics, vol. 31, L25-L30, 1998. In one technique, disclosed by Das et al., “Dielectrophoretic Segregation of Different Cell Types on Microscope Slides,” Anal. Chem. Can 1, 2005, vol. 77, pp. 2708-2719, incorporated by reference herein, a glass slide is patterned with an electrode array in which the electric field frequency decreases in one direction along the length of the slide, which in turn results in a variation of generated DEP forces along the length of the slide. For other examples of the use of DEP particle manipulation via a traveling wave, reference is made to Hagendorn, et al., “Traveling-wave dielectrophoresis of microparticles,” Electrophoresis, vol. 12, pp. 49-54, 1992 and Talary et al., “Electromanipulation and separation of cells using traveling electric fields,” J. Phys. D: Appl. Phys., vol. 29, pp. 2198-2203 (1996), the entire contents of both of which are incorporated by reference herein.

DEP has been used in the separation of viable yeast from non-viable yeast and of other micro-organisms such as Gram-positive bacteria from Gram-negative bacteria, and to remove human leukemia cells and other cancer cells from blood. The use of DEP for separating differing cell types in a device wherein electrode arrays are used to create the non-uniform electric field is disclosed, for example, in U.S. Pat. No. 6,790,330 B2, which issued on Sep. 14, 2004; U.S. Pat. No. 6,641,708 B1, which issued on Nov. 4, 2003; and U.S. Pat. No. 6,287,832 B1, which issued on Sep. 11, 2001, the entire disclosures of which are incorporated by reference herein.

Another more recently developed particle manipulation technique combines the use of DEP force with the concept of optical tweezers. In this regard, the technique is called “optoelectronic tweezers,” and operates by applying an optically activated DEP force to attract or expel a plurality of small particles in a liquid (e.g., aqueous) medium. In contrast to optical tweezers, optoelectronic (OE) tweezers can use a low power incoherent light source, for example, on the order of 1 μW/cm2, instead of the high intensity laser used by optical tweezers. By way of example, optoelectronic tweezers can utilize a light source that has a power approximately ten orders of magnitude less than that of the high intensity lasers typically employed in optical tweezers. In the optoelectronic tweezers technique, a liquid suspension containing various particles, e.g., cells, can be sandwiched between a patternless photoconductive surface and another patternless planar electrode, and can be subjected to a non-uniform electric field generated by projecting the low power incoherent light source onto the photoconductive surface. The non-uniform electric field creates a dielectrophoretic force which acts on the particles such that the particles can be attracted by or repelled from the illuminated area depending upon, among other things, the particles' dielectric properties.

For further explanation of the operation principles of optoelectronic tweezers, including various devices and techniques employing those principles, reference is made to Pei Yu Chiou et al., “Massively Parallel Manipulation of Single Cells and Microparticles Using Optical Images,” Nature, vol. 436:21, July 2005, pages 370-372; WO 05/100541 A2, entitled “Optoelectronic Tweezers For Microparticle And Cell Manipulation,” which claims priority to U.S. Provisional Application No. 60/561,587, filed on Apr. 12, 2004; U.S. application Ser. No. 10/979,645, entitled “Surface Modification For Non-Specific Adsorption Of Biological Material,” filed Nov. 1, 2004, in the name of Aldrich Lau; and U.S. Provisional Application No. 60/692,528, entitled “Optoelectronic Separation of Biological particles: Separation of Dye-labeled DNA, RNA, Proteins, Lipids, Terpenes, and Polysaccharides,” filed Jun. 30, 2005, in the name of Aldrich Lau, the entire contents of each of which are incorporated by reference herein.

Although such optoelectronic manipulation chambers can be useful in biological applications, certain surfaces of the chamber, such as the electrode surfaces, have a tendency to non-specifically adsorb biological material such as cells, proteins, and nucleic acids, inter alia, which can prevent the biological material from being sorted effectively, and could also foul the electrodes. Such non-specific adsorption can occur whether the surface of an electrode is exposed to the biological material directly or the protective layer, for example, silicon nitride, silicon dioxide, or a polymer, adjacent to the conductive electrode material are exposed to the same. Accordingly, it can be desirable to reduce or eliminate non-specific adsorption of biological material in connection with the use of optoelectronic manipulation chambers. Moreover, it can be desirable to provide a technique that permits surface modification of the device, for example, to alter nonspecific and/or specific adsorption. Surface modification of the device can be beneficial to reduce or enhance nonspecific adsorption of, for example, proteins, lipids, cells, and/or other biological material.

It can also be desirable to improve upon devices that utilize optoelectronic tweezers principles in order to manipulate cells. For example, it can be desirable to provide a device that improves robustness, and/or enables operation at a relatively low AC frequency or via direct current.

### SUMMARY

In accordance with the invention, there is disclosed a device for manipulating a biological material, the device comprising: at least one electrode and a photoconductive material configured to receive the biological material; and a light source configured to illuminate the photoconductive material so as to modulate an electric field, wherein the electric field is configured to manipulate the biological material; wherein a surface of the at least one electrode and/or the photoconductive material is modified with at least one of a carboxylic moiety, an amino moiety, a poly(ethylene glycol) moiety, a polymer of poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), and a poly((meth)acrylamide) derivative.

In an aspect, there is also disclosed a method of manufacturing a modified surface for use in a manipulation chamber, comprising: providing a surface to be modified; and bonding to the surface at least one of a carboxylic moiety, an amino moiety, a poly(ethylene glycol) moiety, a polymer of (poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), and a poly((meth)acrylamide) derivative.

In another aspect, there is disclosed a method of reducing adsorption of a biological material in a manipulation chamber, comprising: providing a first electrode and a second electrode; and providing a photoconductive material, and optionally a transparent layer formed over the photoconductive material; wherein a surface of at least one of the first electrode, the second electrode, the photoconductive material, and optionally the transparent layer is modified with at least one of a carboxylic moiety, an amino moiety, a poly(ethylene glycol) moiety, a polymer of (poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), and a poly((meth)acrylamide) derivative; and providing a biological material to be sorted between the first and second electrodes.

In an aspect, there is additionally disclosed a method of increasing adsorption of a biological material in a manipulation chamber, comprising: providing a first electrode and a second electrode; providing a photoconductive material, and optionally a transparent layer formed over the photoconductive material; wherein a surface of at least one of the first electrode, the second electrode, the photoconductive material, and optionally the transparent layer is modified with a carboxylic moiety, which is further modified with at least one of a poly((meth)acrylamide), and a poly((meth)acrylamide) derivative; and providing a biological material to be sorted between the first and second electrodes.

In yet another aspect, there is disclosed a method of manipulating a biological material, comprising: providing a surface; and modifying at least a portion of surface so that it selectively adsorbs at least a portion of the biological material when the surface is at a temperature above a predetermined temperature, and does not adsorb at least a portion of the biological material when the surface is at a temperature below the predetermined temperature.

Further, in an aspect, there is disclosed a plurality of discrete areas on s surface of at least a portion of a first electrode, a second electrode, a photoconductive material, and optionally a transparent layer grafted with at least one of a carboxylic moiety, an amino moiety, a poly(ethylene glycol) moiety, a polymer of (poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), and a poly((meth)acrylamide) derivative.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

It is to be understood that both the foregoing general description and the following detailed description are exemplary and explanatory only and are not restrictive of the invention, as claimed.

The accompanying drawings, which are incorporated in and constitute a part of this specification, illustrate several exemplary embodiments of the disclosure and together with the description, serve to explain certain principles.

### BRIEF DESCRIPTION OF THE DRAWINGS

FIG. 1 is a side view of an exemplary aspect of an optoelectronic manipulation chamber;

FIGS. 2A-11 show exemplary reactions for modifying a surface in accordance with the present disclosure;

FIGS. 12-16 illustrate process steps for fabricating a photoconductive electrode in accordance with the present disclosure;

FIG. 17A shows a top view of an arrangement of photoconductor electrodes during an exemplary process in accordance with the present disclosure;

FIG. 17B shows a side view of the arrangement shown in FIG. 17A;

FIG. 17C shows an end view of the arrangement shown in FIG. 17A;

FIGS. 18-21 illustrate exemplary processes utilizing a surface initiator in accordance with the present disclosure; and

FIG. 22 illustrates an exemplary device comprising a copper or aluminum tape as an electrical contact in accordance with the present disclosure;

FIGS. 23-24 illustrate exemplary methods for making a device in accordance with the present disclosure.

### DETAILED DESCRIPTION

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose. In the event that one or more of the incorporated literature and similar materials differs from or contradicts this application, including but not limited to defined terms, term usage, described techniques, or the like, this application controls.

The term “photoconductive material” as used herein refers to a material that has different electrical conductivity properties when it is in a dark state than when it is in an illuminated state. For instance, the photoconductive material can be an insulator in a dark state and a conductor in an illuminated state.

The term “surface modifier” as used herein refers to compounds capable of chemically modifying a surface to impart a desired characteristic, such as adhesion or non-specific adsorption, to the surface.

The term “glass” as used herein refers to any transparent or translucent material. An example of glass is spin-on-glass (SOG). Commercially available examples of SOG include ACCUGLASS® (Honeywell, Electrical Materials, Sunnyvale, Calif.), which includes T-03AS (dielectric constant at 1 MHz of 6-8, and refractive index at 633 nm of 1.43), P-5S (dielectric constant at 1 MHz of 4.7, and refractive index at 633 nm of 1.48), and T-12B (dielectric constant at 1 MHz of 3.2, and refractive index at 633 nm of 1.39). Another example is LUDOX® (Grace Davison, Columbia, Md.) which is a colloidal suspension of spherical silica particles for surface coating. Upon drying, the hydroxyl groups on the surface of the particles condense to form siloxane bonds (Si—O—Si) resulting in coalescence and interbonding.

The term “non-specific adsorption” as used herein refers to indiscriminate adsorption, unintentional adsorption, or undesirable adsorption of an object of interest, or an undesirable object, to a random location, unknown location, or unwanted location on an electrode or proximate to the electrode.

As described above, DEP (dielectrophoresis) is the motion imparted on uncharged particles, for example, through a solution, as a result of polarization induced by nonuniform electric fields, whereas electrophoresis (EP) is the migration of particles through a solution under the influence of an applied electric field by virtue of the particle's charge. It is envisioned that both optoelectronically induced DEP and EP can be used to manipulate particles in accordance with the various exemplary aspects of the disclosure.

It should be noted that sizes and configurations of various structural parts and materials used to make the parts disclosed herein are illustrative and exemplary only. One of ordinary skill in the art would recognize that those sizes, configurations, and materials can be changed to produce different effects and/or desired characteristics. Further, although many of the embodiments described herein are discussed in conjunction with using optoelectronic manipulation principles for applications relating to cellular analysis and other cell biology applications, it should be understood that the exemplary techniques and devices disclosed herein can be used for other applications wherein the manipulation of small particles is desirable, such as, for example, clinical diagnostics, drug discovery, environmental monitoring of biological particles and non-biological particles (e.g., detection of viral, bacterial, or protozoan entities in water samples and detection of non-biological particulates in water samples), characterization and/or isolation of non-cell microparticles (e.g., microsphere sizing or chemical/electrical characterization), and/or other applications wherein manipulation (including identification, sorting, separating, moving, quantitating, characterizing, etc.) of small particles can be desired. Yet other applications can include, but are not limited to, manipulation, including separation, of dye-labeled DNA, RNA, proteins, lipids, terpenes, glycoconjugates, and polysaccharides, for example.

As used in this application, the terms “small particles” and “biological material” can be used interchangeably and include micro- and/or nano-particles, for example, particles having dimensions on the order of a few microns or a few nanometers. In the context of biological fluid analysis and/or handling, the terms can include cells, cell aggregates, cell organelles, stem cells, nucleic acids, bacteria, protozoans, viruses, and other biological particles.

Reference will now be made in detail to several exemplary aspects of the disclosure, which are illustrated in the accompanying drawings. Wherever possible, the same reference numbers will be used throughout the drawings to refer to the same or like parts.

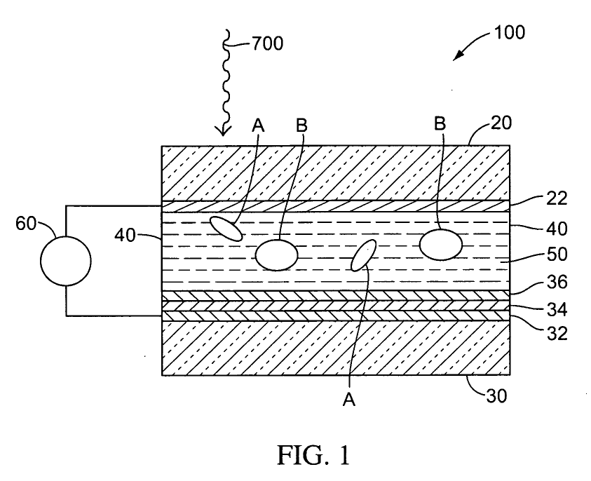

FIG. 1 schematically illustrates an exemplary aspect of a manipulation chamber which relies on optically activated DEP (O-DEP) particle manipulation for use in optoelectronic scanning and other manipulation techniques, in accordance with various exemplary aspects of the present disclosure. The optically activated DEP manipulation chamber also is referred to herein as an optoelectronic manipulation chamber. The chamber 100 can include two substrates, 20 and 30, disposed in a spaced relationship so as to be configured to contain therebetween a sample for analysis. By way of example the substrates 20 and 30 can be spaced from each other by a distance ranging from about 10 microns to about 200 microns. The edges of the electrode units can be provided with a seal 40, such as, for example, a gasket (e.g., a rubber gasket such as a silicon rubber gasket, or a fluorinated elastomer (VITON®) gasket), an adhesive (e.g., a pressure sensitive adhesive (PSA)), and/or other sealing mechanisms, so as to contain the sample liquid in the chamber.

The chamber also can be provided with various ports and/or valves (not shown in FIG. 1), for example, input and output ports and/or valves, to allow for introduction of sample or other materials, including manipulation tools, for example, to the chamber, flushing of sample and/or particles from the chamber, and/or collection of particles from the chamber. By way of example, the input and output ports of the chamber can form an interface with instrumentation separate from the chamber via O-rings in a clamping fixture or via resealable elastomeric material, such as, for example, a septum. The septum can permit a needle to pass therethrough for sample addition and/or removal. Instrumentation which can interface with the chamber can include, but is not limited to, valves, such as, for example, pinch or solenoid valves, and/or pumps, such as, for example, a peristaltic pump or a syringe pump for microfluidic control (e.g., sample introduction and collection).

The cavity 50 can comprise, for example, a liquid suspension containing a plurality of small particles of differing types (for example, differing cell types) labeled A and B in the exemplary embodiment of FIG. 1. It should be understood that the liquid suspension can contain any number of differing types of particles and the use of two particle types A and B herein is for ease of reference and explanation. According to exemplary aspects, the small particles can be suspended in an aqueous medium, such as, for example, a phosphate buffer, a phosphate-buffered saline (PBS), which can contain about 1% bovine serum albumin (BSA), for example, a saline solution having a pH ranging from about 6.5 to about 8.5 and a conductivity ranging from less than about 10 mS/m to several hundred mS/m, a potassium chloride solution, or other suitable mediums, such as mediums that are biologically compatible with cells and iso-osmotic. By way of example, the medium can also comprise a HEPES (N-2-Hydroyxyethylpiperazine-N′-2-ethanesulfonic acid) buffer, sugars, such as mannitol, sorbitol, trehalose, sucrose, or dextrose, for osmotic stability, and/or solutes for modifying the medium permittivity, such as, for example, ∈-amioncaproic acid, and/or solutes for modifying the medium density, such as, for example, OptiPrep or Nycodenz (Axis Shield).

According to exemplary aspects, the first and second substrates 20 and 30 can be made of a transparent, insulating material including, but not limited to, glass, silica, quartz, plastic, ceramic, highly transparent polymers such as poly(cyclic olefin), polymethylmethacrylate (PMMA), polycarbonate, polystyrene, and/or copolymers thereof, or other suitable transparent and insulating material. Further, in an exemplary aspect, the surfaces of the substrates 20 and 30 facing the chamber interior can be modifiable and/or provided with an adhesive promoter so as to enhance adhesion of the electrode layers thereon. Depending on where a light source is to be positioned for illuminating the photoconductive material, one or both of the substrates 20 or 30 need not be transparent. For example, in the example shown in FIG. 1, wherein the light source is positioned so as to transmit light through the first substrate 20, the second substrate 30 need not be made of a transparent material, and vice versa if the light source is positioned so as to transmit light through the second substrate 30.

The first substrate 20 can comprise a transparent electrode 22 facing the cavity 50. The second substrate 30 also can comprise an electrode 32, such as, for example a metal electrode. In various alternative aspects, the substrate 30 adjacent the electrode 32 can be nontransparent and constructed of any material that can withstand the processing conditions for deposition of a photoconductive material. The electrode 22 and the electrode 32 may be electrically coupled to a power supply 60, which may be AC or DC.

In various exemplary aspects, the transparent electrode 22 can be, for example, gold, indium tin oxide (ITO), or other suitable transparent electrode material. The term “transparent” in this context means that at least some light can pass through the layer. For example, due to a nonuniform deposition of the electrode layer on the substrate, at least some regions can have no electrode material deposited thereon or a very thin layer thereof, and light can pass through those regions. In an exemplary aspect, the transparent electrode can be such that from approximately 20% to approximately 95% of the incident light can pass through the electrode layer. In an exemplary embodiment, which is described below in more detail, the electrode 22 can be a transparent gold electrode (TGE) with a PEGylated surface.

The electrode 32 can be a transparent or nontransparent electrode. By way of example, the electrode 32 can be made of indium tin oxide, gold, aluminum, copper, nickel, chromium, a metal alloy, or any other suitable conductive material. In the case where electrode 32 is transparent, those skilled in the art would be able to determine the thickness of electrode 32 in order to maximize the transparency, percentage of incident light to pass through, and conductivity.

The power supply 60 can be AC or DC. According to various exemplary aspects, an AC current having a relatively high frequency ranging from approximately 1 kHz to 10 MHz can be used. Alternatively, the AC current can have a relatively low frequency ranging from less than approximately 10 Hz to less than approximately 1 kHz. One of ordinary skill in the art would understand that any frequency or range of frequency can be used.

A layer of photoconductive material 34 can be provided over the electrode 32 so as to close the circuit. The photoconductive material can be amorphous silicon (α-Si:H), but can also be made of single crystal silicon, amorphous selenium, polyferrocenylsilane, or other photoconductive materials as known in the material science arts. A tie layer (not shown) can be used as an interface between the electrode 32 and the photoconductive material 34. The tie layer can serve as a compatibilizer, thereby providing better adhesion of the photoconductive material 34 to the electrode 32. In an exemplary aspect, the tie layer can be an n+ doped amorphous silicon layer.

In an exemplary aspect, the photoconductive material 34 can be separated from the cavity 50 by a transparent material layer 36, such as, for example, a polymer dielectric, insulating Spin-on-Glass (SOG), a semiconductive SOG, a coalesced colloidal silica (LUDOX®) layer, a semiconductive transparent film, a silicon nitride film, a silicon dioxide. In various exemplary aspects, 36 may be silicon dioxide layer with its surface PEGylated, a silicon dioxide layer with surface-grafted poly(acrylamides). In an exemplary aspect, the second substrate 30 can be provided with a tie layer, and a PEGylated silicon dioxide transparent layer 36.

In an exemplary aspect, the transparent layer 36 can be a layer of silicon dioxide (SiO₂) from less than 1 nm to 100 nm thick, and can be deposited onto the photoconductive material 34 either by chemical means, such as a Piranha solution treatment, oxygen plasma treatment, or by plasma-enhanced chemical vapor deposition (CVD). In various exemplary aspects, as will be explained below, the surface of the silicon dioxide transparent material layer 36 can be modified by PEGylation, or by grafting poly(acrylamides) thereon.

In an exemplary aspect, the transparent layer 36 can be silicon dioxide (natively grown or vapor deposited). The transparent layer 36 can be pre-treated to clean and/or to increase the surface density of silanol groups. For example, the pre-treatment can include a procedure whereby the transparent layer 36 can be first rinsed consecutively with acetone, water, and acetone, followed by a 1:1:4 v/v solution of 29% NH₄OH, 30% H₂O₂, and deionized water, and followed by a 1:1:4 v/v solution of 38% HCl, 30% H₂O₂, and deionized water. It should be noted, however, that such pre-treatment is not required.

In various exemplary aspects, the transparent electrode 22 can be indium tin oxide (ITO) and can be pre-treated to clean and/or to increase the surface density of hydroxyl groups prior to chemical surface modification. The pre-treatment can include a procedure whereby the ITO surface 22 can be first rinsed consecutively with acetone, methanol, and water, followed by a 1:1:4 v/v solution of 29% NH₄OH, 30% H₂O₂, and deionized water.

In accordance with various exemplary aspects, a light source 700, such as a scanning light beam, can be used to illuminate a portion of the photoconductive material 34 and thereby close the circuit between the transparent electrode 22 and the electrode 32. Transmitting the light onto the photoconductive surface 34 converts the illuminated region of that surface to a virtual electrode, thus generating (e.g., modulating) a nonuniform electric field and corresponding DEP force that acts upon the particles A and B in the sample layer 50. A nonuniform electric field is modulated as a result of the difference in areas of the electrode 22 and the virtual electrode created by the illuminated region of the photoconductive surface 34. Due to the differing dielectric properties and size of each particle type A and B, the differing particle types A and B experience differing forces, including DEP forces, so as to allow manipulation of the particles as is explained further below.

A variety of light sources can be used to illuminate the manipulation chamber including, but not limited to, LEDs, phosphor coated LEDs, organic LEDs (OLED), phosphorescent OLEDs (PHOLED), inorganic-organic LEDs, LEDs using quantum dot technology, and LED arrays. Alternatively, suitable light sources can include, but are not limited to, white light sources, halogen lamps (e.g., xenon or mercury arc lamps), lasers, solid state lasers, laser diodes, micro-wire lasers, diode solid state lasers (DSSL), vertical-cavity surface-emitting lasers (VCSEL), thin-film electroluminescent devices (TFELD), filament lamps, arc lamps, gas lamps, and fluorescent tubes. According to various exemplary aspects, the light source can be an incoherent light source. In various exemplary aspects, the incident light can range from visible to UV range and can enable visualization through inherent fluorescence characteristics of some particles (e.g., cells). The light source can operate at a power from about 0.01 μW/cm² to about several hundred W/cm², for example. By way of example, suitable mechanisms for causing the light source to scan include, but are not limited to, galvanometers and digital light projectors (DLP).

A variety of materials can be used for the various elements of the manipulation chamber, and the various layers may be treated (e.g. via surface modification) so as to alter performance of the chamber. By way of example, in various exemplary aspects, one or more surfaces of the chamber can be surface modified to either reduce non-specific adsorption of cells (e.g., using poly-I-lysine can be used to modify the surface) or enhance selective adsorption of particular particle (e.g., cell) types (e.g., using antibodies, lectins, ligands, smart polymers). In various exemplary aspects, chemical functionalities, including but not limited to, amino or carboxylic moieties, can be covalently attached onto different areas of a surface such that bioconjugation can be performed in subsequent steps to anchor biomolecules (e.g., antibodies, phage proteins, biotins, streptavidins, ligands, smart polymers) to bind cells. Further, differing areas on a surface can be subject to differing modifications such that different cell types can bind to the different areas.

Moreover, although in various aspects described herein, the manipulation chamber is disclosed as comprising approximately planar substrates sandwiching a spacer (e.g., a seal such as PSA), sample liquid, and material layers, it should be understood that various other configurations may be envisioned and are considered within the scope of the disclosure. In general, any device configuration may be utilized such that a light source illuminating a photoconductive surface generates a nonuniform electric field and a corresponding DEP force on the particle solution within the manipulation device.

The present disclosure reduces or eliminates the problem of non-specific adsorption of biological material on the electrode units of a manipulation chamber by chemically modifying the surfaces, for example of the electrodes and/or the transparent material layer over the photoconductive layer. In particular, the disclosure includes methods for chemically bonding a poly(ethylene oxide) (PEO) or poly(ethylene glycol) (PEG) moiety (a process hereinafter referred to as “PEGylating”) to surfaces, such as electrodes and silicon dioxide, and manipulation chambers constructed using PEGylated surfaces. Further, the present disclosure includes methods for modifying surfaces with poly((meth)acrylamide) and/or its derivatives thereby enabling the binding (capture) and release of biological material (e.g., cells) to the modified surfaces.

PEGylation can occur by bonding a PEO or PEG moiety comprising from about 5 to about 1000 repeating units, for example, from about 6 to about 300, or, for example, from about 10 to about 200 to a surface to be modified. Those skilled in the art would be able to determine the number of repeating units of the PEO or PEG moiety to achieve desired surface features.

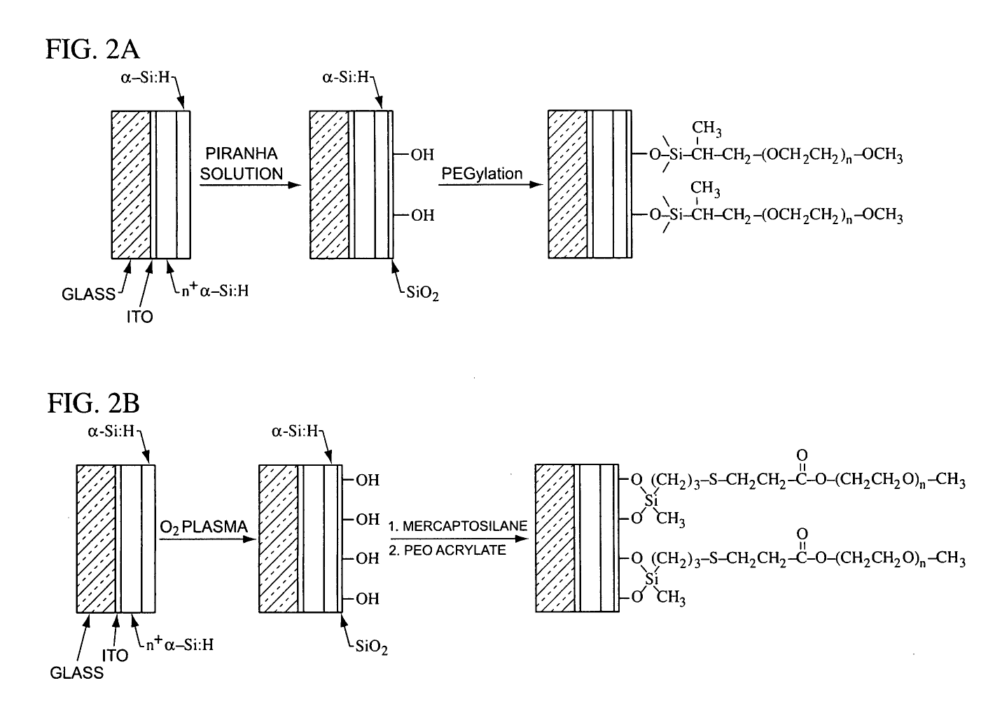

Various reactions can be effected to PEGylate a surface in accordance with the disclosure. For purposes of illustration only, examples of such reactions are provided with reference to FIGS. 2A-11; however, such PEGylation can be achieved by many other reactions which, although not specifically discussed herein, are within the scope of the invention.

In the exemplary approach shown in FIG. 2A, the photoconductive layer can comprise a n+ α-Si:H (doped amorphous silicon) tie layer (adhesion promoter and electric contact) with a α-Si:H (amorphous silicon) photoconductive layer deposited thereon. A Piranha solution can be applied to the surface of the photoconductive layer of step (i) in FIG. 2A, which can result in growing a skin (protective) layer of silicon dioxide with silanol hydroxy groups (OH) attached to the surface, as depicted in step (ii). Silicon dioxide can then be subjected to a PEGylation process using 2-[methoxy(polyethyleneoxy)propyl]trimethoxysilane (obtainable from Gelest, Inc.), which yields the configuration depicted in step (iii). It should be noted that n in the PEG groups in step (iii) can range from about 2 to about 300. As n increases, nonspecific adsorption can be reduced.

A second exemplary approach achieves PEGylation of a photoconductor layer via Michael type addition reaction as illustrated in FIG. 2B. In this approach, cleaning and enhancing the density of silanol hydroxyl groups on the photoconductive surface can occur in a single step within a vacuum chamber. As depicted herein, the structure can be substantially the same as described with reference to step (i) in FIG. 2A above, having a glass substrate with electrode and photoconductor layers thereon. Within a single vacuum chamber process, the substrate shown in FIG. 2B (i) can be subjected to an oxygen plasma treatment. The oxygen plasma cleans the surface of the amorphous silicon and at the same time grows a skin (protective) layer of silicon dioxide on the surface, as depicted in FIG. 2B (ii). The resulting skin layer of silicon dioxide can then be subjected to a mercaptosilanization with (3-mercaptopropyl)methyldimethoxysilane (obtainable from Gelest, Inc.), which results in the implantation of surface mercapto groups thereon. The mercapto groups can be further reacted with poly(ethylene glycol) methyl ether acrylate, PEO acrylate (obtainable from Aldrich Chemical), to yield a PEGylated photoconductive layer, as depicted in FIG. 2B (iii). As with FIG. 2A, n in the PEG groups in step (iii) can range from about 2 to about 300. In various exemplary aspects, a mixture of PEO acrylates with various n values can be used.

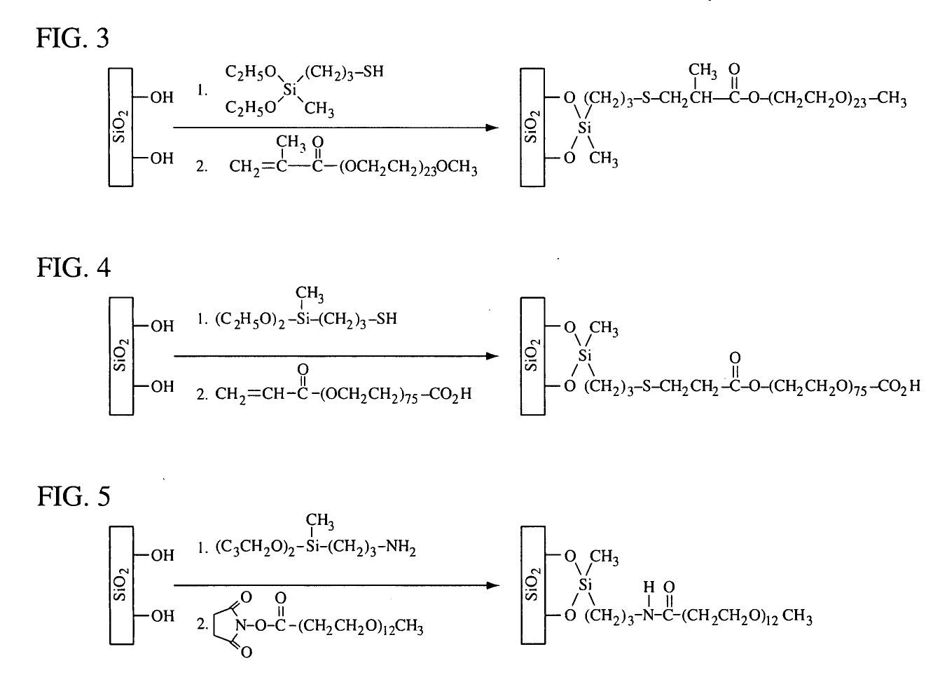

In various exemplary aspects, a layer of silicon dioxide can be deposited onto the photoconductor surfaces as by plasma-enhanced chemical vapor deposition (CVD). Different surface areas of the silicon dioxide can be PEGylated via a Michael addition reaction, as shown in FIG. 3. FIG. 4 illustrates PEGylation via a Michael addition reaction similar to that shown in FIG. 3. This resultant modified surface can readily be subjected to bioconjugation via the carboxylic acid groups. Biomolecules, can include but are not limited to, biotins, streptavidins, enzymes, proteins, phage proteins, antibodies, glycoconjugates, and ligands can be immobilized ionically or covalently thereon. For examples of bioconjugation of biomolecules, reference is made to Greg T. Hermanson, “Bioconjugation Techniques,” Academic Press, 1996, the disclosure of which is hereby incorporated by reference.

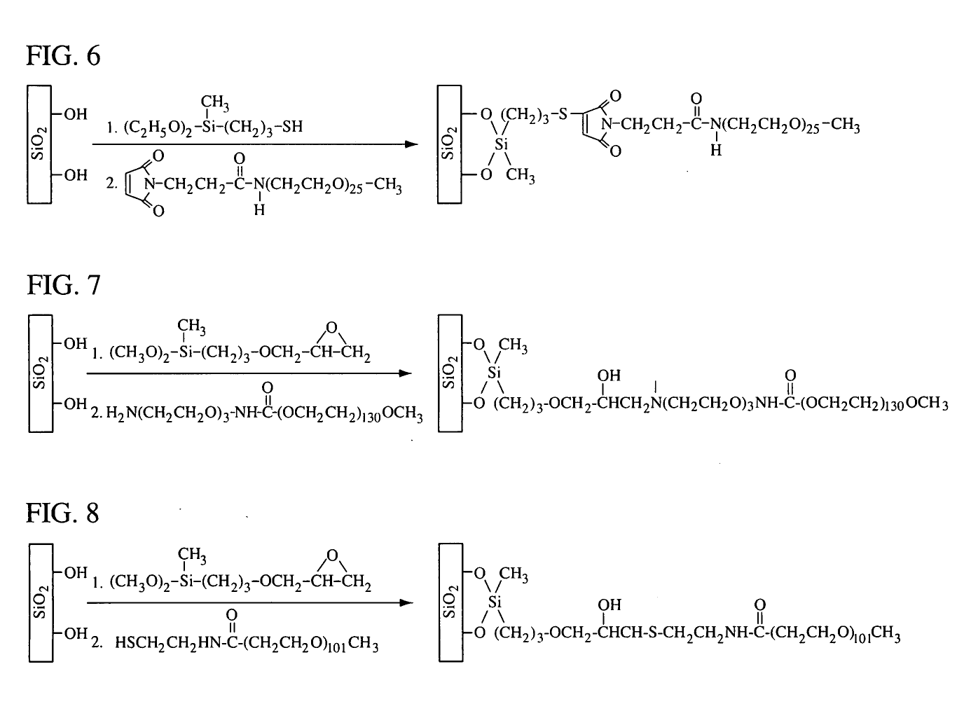

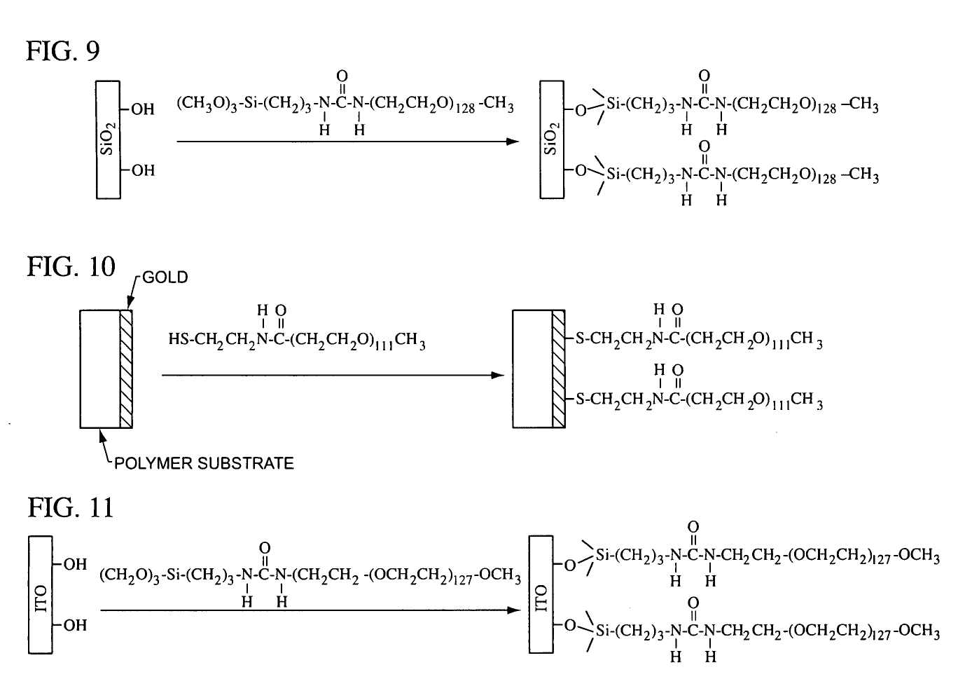

FIG. 6 shows a two-step PEGylation reaction based on PEG-N-hydrosuccinimide ester (PEG-NHS ester). In FIG. 6, the surface is PEGylated in a two-step reaction based on maleimido-PEG. FIG. 7 illustrates PEGylation by first reacting the surface with glycidylalkoxysilane, and then reacting the intermediate structure with amino-PEG. In FIG. 8, the surface is PEGylated in a two-step reaction based on mercapto-PEG. FIG. 9 is yet another exemplary reaction for PEGylating a silicone dioxide layer.

FIGS. 17A-C shows a reaction in which a surface of the manipulation chamber can be PEGylated in a solvent-free environment, according to another exemplary aspect. FIG. 10 illustrates an example in which a gold electrode (transparent gold electrode) can be PEGylated with mercapto-functionalized poly(ethylene glycol) (molecular weight 5723 Da, obtained from Nektar) in a neat reaction. Similarly, FIG. 11 illustrates an example in which an ITO electrode can be PEGylated in a neat reaction. One of ordinary skill in the art would appreciate the benefits of a neat reaction including lower consumption of materials, less process steps, higher resultant yield, and easier scalability, for example.

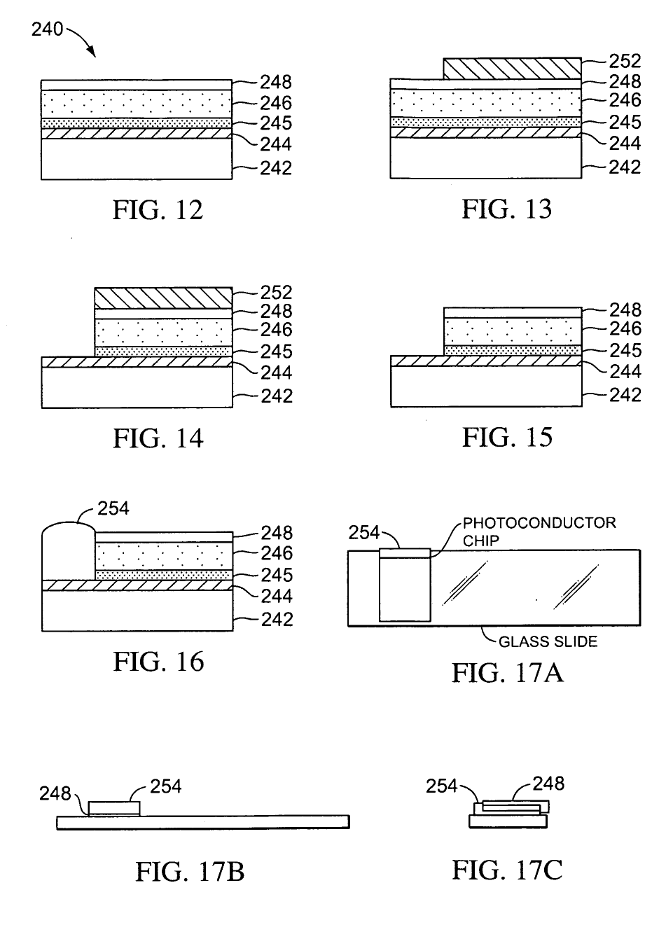

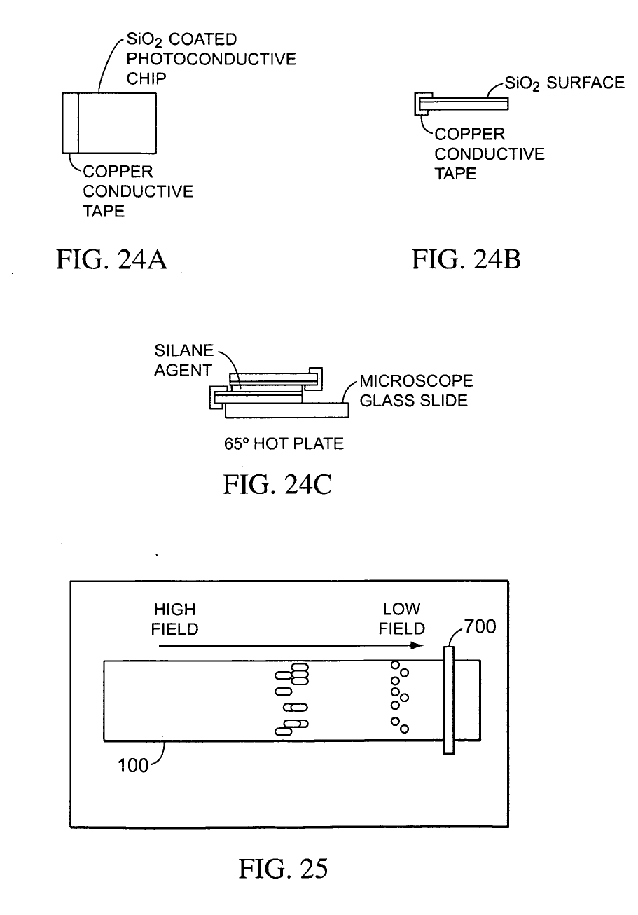

An exemplary process for fabricating PEGylated photoconductive electrodes in accordance with the invention will now be discussed with reference to FIGS. 12-16 and 22-24.

In the example shown in FIG. 12, a photoconductive electrode 240 includes a PYREX® glass substrate 242 having a thickness of about 0.85 mm, a conductive layer 244 made either of ITO and having a thickness of approximately 200 nm or gold and having a thickness of 10-200 nm, an n+ doped α-Si:H tie layer 245 formed to a thickness of about 50 nm, a photoconductive layer 246 made of α-Si:H having a thickness of about 1000 nm, and a SiO₂ layer 248 formed by vapor deposition to a thickness of approximately 5-10 nm. A masking layer 252 is then layered over the SiO₂ surface, as seen in FIG. 13. The mask is patterned to expose regions of the photoconductor unit on which an electrode contact is to be added. Next, reactive ion etching (RIE) is performed to remove the layers under the exposed regions of the photoconductor unit until the conductive layer 244 is exposed, as illustrated in FIG. 14. The mask is then removed, as shown in FIG. 15, and an electrical contact 254 is provided into the void created by the RIE, as can be seen in FIG. 16.

The electrical contact 254 is a conductive silver epoxy (EPO-TEK E2101, obtained from Epoxy Technology, Billerica, Mass.). In an alternative embodiment, the electrical contact 254 can be gold metal, which can be formed by vapor deposition prior to removing the masking layer. It is noted that Piranha solution will dissolve silver epoxy, but is non-reactive with respect to gold; accordingly, if the electrode is to be treated with Piranha solution prior to PEGylation, for example, to increase the density of silanol groups on the SiO₂ surface, then gold should be used as the electrical contact material.

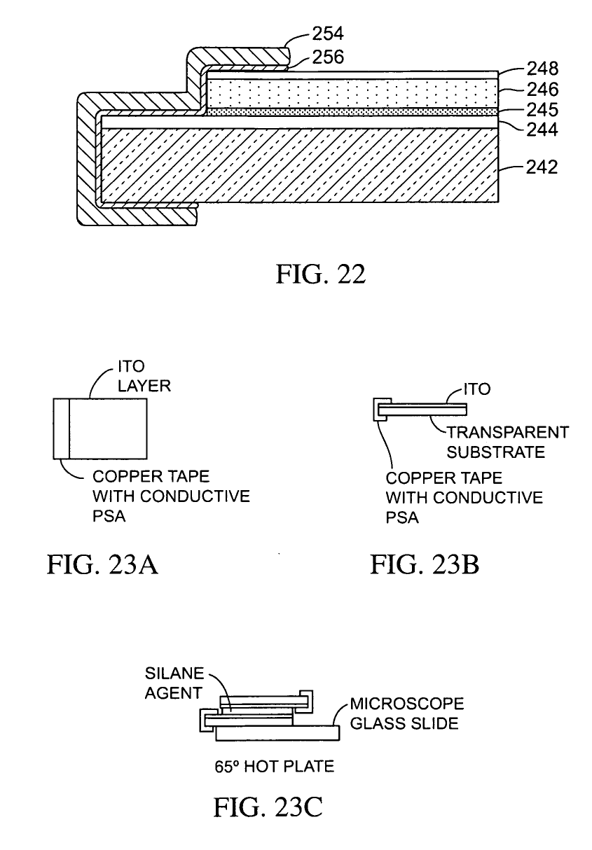

Other alternatives for the electrical contact 254 are copper or aluminum tapes (CCH-36-101 and CCJ-36-201, respectively, obtainable from Chomerics, Woburn, Mass.) as illustrated in FIG. 22. The tape has a layer of pressure sensitive, conductive adhesive 256 on one surface, thus allowing it to be applied onto the ITO or gold surface 244 after reactive ion etching and the removal of mask, after pre-treatment or after PEGylation. FIGS. 23A-C and 24A-C illustrate exemplary methods for fabricating a photoconductor chip having a PEGylated ITO electrode or PEGylated SiO₂ surface, respectively.

After addition of the electrical contact, the electrode structure can optionally be pre-treated as discussed above to enhance the surface density of silanol groups and then PEGylated using one of the exemplary methods discussed above. The set up for the exemplary PEGylation process is illustrated in FIG. 17A showing the top view thereof, FIG. 17B showing the side view thereof, and FIG. 17C showing the end view thereof. Here, the electrode chips are stacked with the silicon dioxide surfaces facing each other and the electrical contacts 254 exposed. The electrode chips can be placed on a hot plate or an oven to melt the PEG-silane agent 248 at a temperature range of about 60°-65° C., forming a liquid layer in direct contact with the target PEGylation surfaces.

### Example 1

In order to test the effect of PEGylation on cell adhesion, photoconductive substrates were obtained with 5 nm or 10 nm thick SiO₂ layers deposited by CVD. These substrates were PEGylated according to the process described in FIG. 9, and FIGS. 12-17C. DEP manipulation chambers from these PEGylated substrates were prepared as in FIG. 1, using PSA (˜100 um) as the spacer (FIG. 1; 40) between the PEGylated photoconductor and an ITO transparent electrode coated substrate (FIG. 1; 20 and 22). The liquid suspension in the cavity (FIG. 1; 50) contained Hela cells at ˜3×10⁵ cells/mL, in 8.5% sucrose, 0.3% dextrose buffer, and adjusted to ˜2 mS/m conductivity by the addition of DMEM, 10% FBS growth media. DEP force was induced in the device by projection of a ˜100×30 um light pattern (˜635 nm) onto the photoconductor surface and the application of ˜10 Vpp AC voltage (FIG. 1; 60) between the ITO and photoconductor surfaces. For further explanation of the operation principles of the manipulation chamber, reference is made to Pei Yu Chiou et al., “Massively Parallel Manipulation of Single Cells and Microparticles Using Optical Images,” Nature, vol. 436:21, July 2005, pages 370-372. Cell mobility or adhesion was tested by moving the projected light pattern next to a Hela cell and recording the presence (mobility) or absence (adhesion) of cell movement toward the light pattern (positive DEP). Three devices each comprising 5 nm or 10 nm of a SiO₂ layer on the photoconductive surface were tested and approximately 100 cells were tested per device. Table 1 shows the results from these experiments. On average, >95% of Hela cells were mobile on both the 5 nm and 10 nm versions of the device, with the 10 nm devices showing a slightly higher percentage of mobile cells. In contrast, on average, <20% of Hela cells were mobile on un-PEGylated photoconductor surfaces. These data suggest that PEGylation can significantly reduce the non-specific adsorption of cells onto the photoconductor surface.

TABLE 1

| SiO₂ layer (nm) | Mean % Mobile | Std. Dev. |
|---|---|---|
| 5 | 95.4 | 0.90 |
| 10 | 98.4 | 0.52 |

In an aspect, selected regions of a relevant surface(s) can be modified so that desired biological material can be selectively adhered onto the surface(s) according to a specified pattern. For example, through lithographic means, selective adhesion enhancing grafts can be covalently anchored on different regions of a surface of a SiO₂-coated photoconductor, forming a plurality of discretely grafted areas. The plurality of discretely grafted areas can form an array of rows and columns. The grafts can be at least one of a carboxylic moiety, an amino moiety, a poly(ethylene glycol) moiety, a polymer of (poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), a poly((meth)acrylamide) derivative, and any functional groups that allow for bioconjugation. The rest of the ungrafted areas can then be PEGylated in subsequent steps. The size of a discrete area can range, for example, from 1 square micron to several square millimeters. Those with ordinary skill in the art can determine the appropriate size of a grafted area in order to effectively capture (bind) and release certain types of biological material. This technique can provide the ability to control the adhesion of biological material in a manipulation chamber, which greatly expands the utility of the chamber.

As mentioned previously, bioconjugation can be employed to bind biological material to a modified surface having certain chemical functional groups. In this regard, functional groups such as, for example, a carboxylic moiety, an amino moiety, a poly(ethylene glycol) moiety, a polymer of (poly(ethylene oxide) methyl ether) acrylate, a poly(2-hydroxyethyl (meth)acrylate), a poly(N-vinylpyrrolidone), a poly(N-vinylformamide), a poly(N-vinylformamide) derivative, a poly((meth)acrylamide), and a poly((meth)acrylamide) derivative, can be grafted onto an electrode or silylated SiO₂ surface to enable bioconjugation. One of ordinary skill in the art would thus realize that bioconjugation-enhancing functional groups can be grafted onto a surface via many different types of reactions. As such, bioconjugation can be performed on an SiO₂ surface alone, or an SiO₂ surface modified by PEGylation, in accordance with the present disclosure.

In another approach to enhancing selective adhesion of biological material by surface modification, certain derivatives of polyacrylamides, including N-substituted variants of acrylamide or methacrylamide, have been shown to exhibit thermally reversible solubility in water. Examples of such derivatives include N-substituted variants of acrylamide or methacrylamide. At temperatures below the lower critical solution temperature (LCST), such derivatives can be hydrophilic and hence soluble in water. Above the respective LCSTs, the derivatives can be hydrophobic, and hence less soluble in water (Heskins M. and J. E. Guillet. J., Macromol. Sci.—Chem. 1968, A2(8):1441-1455; Okano, T, et al., U.S. Pat. No. 5,284,766,1994).

A few examples of some N-substituted acrylamide compounds exhibiting thermally reversible water solubility and their respective LCST's are shown below.

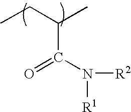

wherein R¹ and R² can represent any of the formulas shown in Table 2:

TABLE 2

| R¹ | R² | LCST (° C.) |
|---|---|---|
| H | Et | 82 |
| H | Et | 73 |
| H | Pro | 22 |
| H | Iso-pro | 32 |
| H | Methoxypropyl | 10 |
| Me | Me | |
| Me | Et | 56-57 |
| Me | Pro | 15 |
| Me | Iso-pro | 25 |
| Et | Et | 25 |
| Et | Et | 32 |
| Et | Et | 36 |

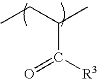

wherein R³ can represent any of the formulas listed in Table 3:

TABLE 3

| R³ | LCST (° C.) |
|---|---|
| 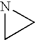 | 57 |
| 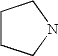 | 56 |
| 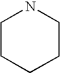 | 5 |

Co-monomers, for example, (meth)acrylic acid, esters of (meth)acrylic acid, (meth)acrylamide, substituted (meth)acrylamides, 2-hydroxyethyl (meth)acrylate (HEMA), N-vinyl pyrrolidone (NVP), other vinyl monomers, and combinations thereof can be incorporated during polymerization compounds to control the hydrophilicity and LCST transition temperature of the final product.

It is contemplated that once a surface has been modified with a polyacrylamide and/or its derivative, one could either increase or decrease adsorption of biological material to the modified surface by increasing or decreasing the temperature of the modified surface to above or below a predetermined temperature.

Thermally reversible adhesion by surface modification with a polyacrylamide with LCST properties can be useful in cell culture research activities, inter alia. For example, at 37° C., the typical growth temperature for tissue culture, surfaces modified with poly(N-isopropylacrylamide) (PIPAAm) is relatively hydrophobic, and can promote attachment of the biological material and spreading thereon (Okano, T, et al., U.S. Pat. No. 5,284,766,1994; Akiyama, Y, et al., Langmuir 2004, 20:5506-5511). At temperatures below the LCST, e.g., below about 10 or about 20° C., the PIPAAm becomes more hydrophilic, and the biological material can lift off the surface with no or reduced trypsin treatment and can be collected as individual cells or as sheets (Kushida, A., et al., J. Biomed. Mater. Res. 1999, 45:355-362; Chen, G., et al., J. Biomed. Mater. Res. 1998, 42:38-44; Okano, T., et al., Biomaterials 1995, 16:297-303). Biological material that has been released from PIPAAm surfaces can contain a more intact extracellular matrix than cells released by trypsin proteolysis, and as such, can exhibit more physiologically relevant behavior during cell passage and collection. (Okano, T., et al., Biomaterials 1995, 16:297-303; Yamato, M., et al., Biomaterials 2000, 21:981-986; Canavan, H., et al., Langmuir 2005, 21, 1949-1955). Thus, it is contemplated that once a surface has been modified with a polyacrylamide and/or it derivative one could either increase or decrease adsorption of biological material to the modified surface by increasing or decreasing the temperature of the modified surface to above or below a predetermined temperature.

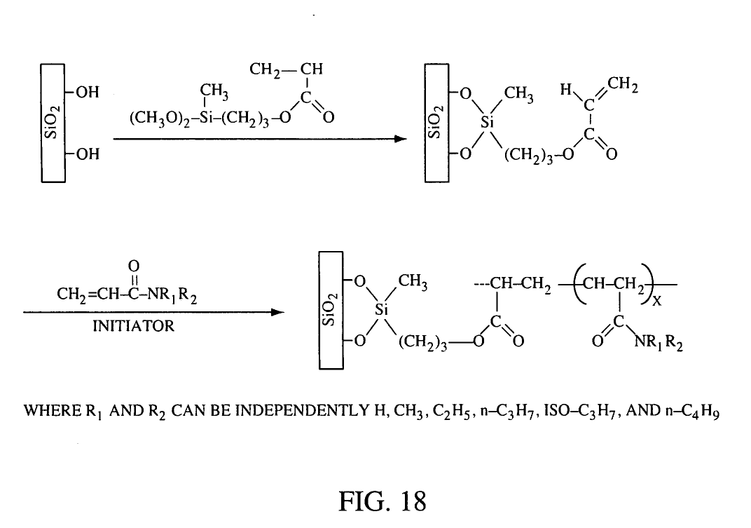

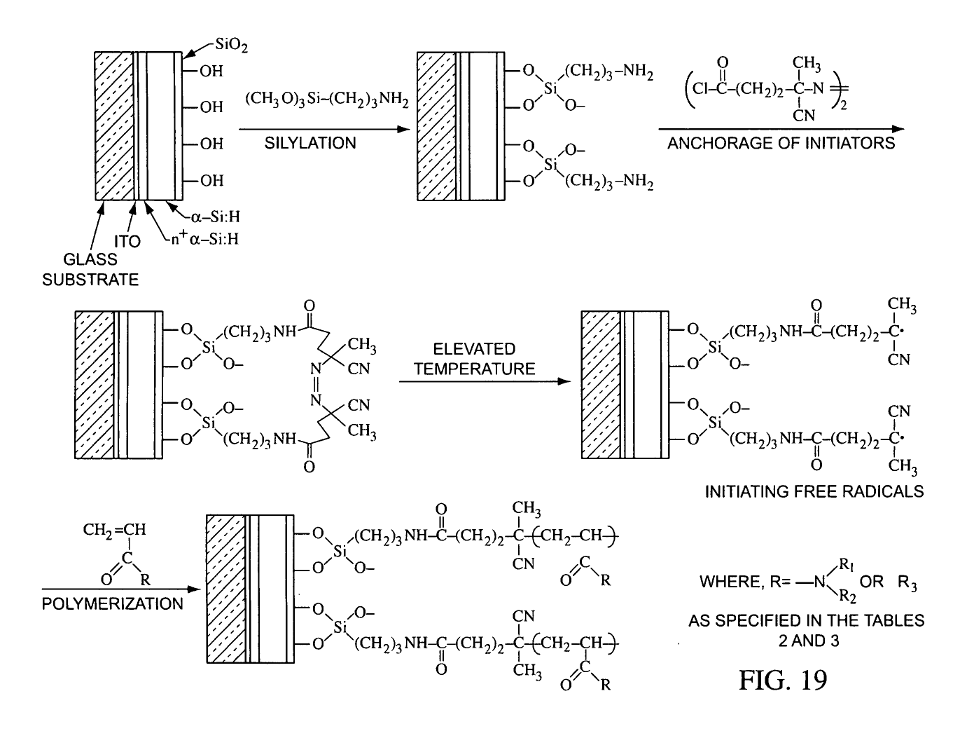

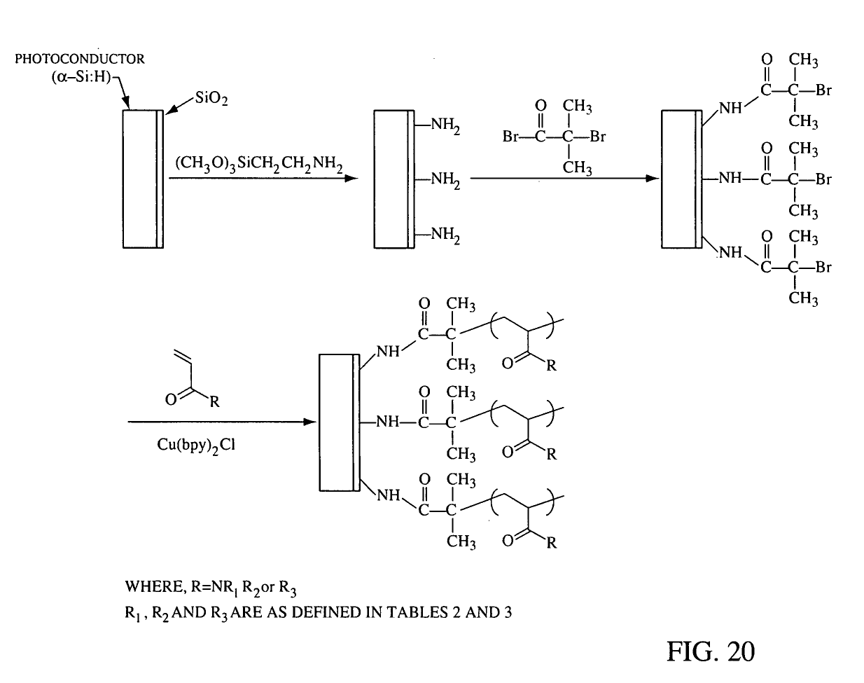

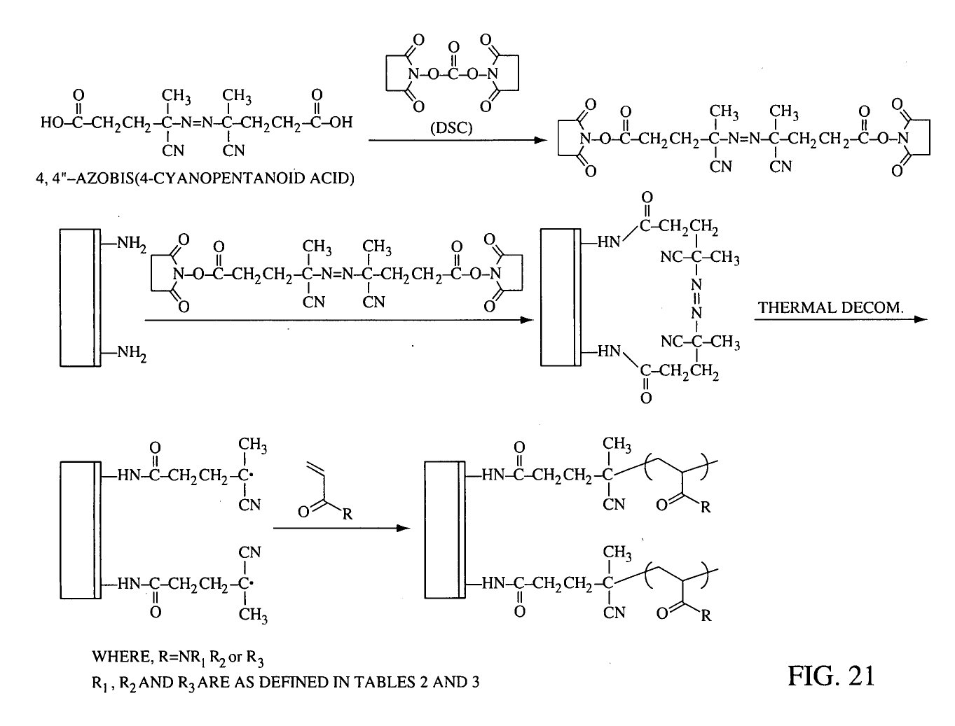

In another aspect, a surface initiator that initiates free radical grafting can be covalently attached to the surface to be modified as shown in FIGS. 18-21. The free radical polymerization can occur on the surface and not in solution thereby providing control in the composition and thickness of the resulting LCST hydrogel. FIGS. 20 and 21 illustrate exemplary reactions for surface grafting poly((meth)acrylamide) and/or its derivatives. In an aspect, vinyl monomers can be replaced with 2-hydroxyethyl (meth)acrylate (HEMA) or N-vinyl pyrrolidone (NVP).

It is contemplated that a manipulation chamber comprising a surface modified with polyacrylamide and/or its derivatives can be used to sort biological material. For example, biological material, such as cells could be sorted by changing the applied field or frequency of an O-DEP line during scanning of a light source across the chamber thereby resulting in “bands” of cells with similar DEP potential. Thus, as shown in FIG. 25, differing types of biological material, such as cells A and B, exhibit differing movement characteristics based on the material's dielectric properties and/or size, for example. Such dielectrophoretic movement characteristics include the displacement (dielectrophoretic displacement) of a type of biological material and the speed of manipulation (dielectrophoretic speed) of the particle type as a result of an applied DEP force resulting from a moving incident light 700 relative to the biological material. Those skilled in the art will understand that various waveforms can be used to control the scanning speed as a function of time, and that the particular function used will depend on, among other things, the scanning application.

After the cells are sorted, the cells could be induced to adhere and spread on to the surface by raising the temperature of the modified surface, e.g., to about 37° C. Once the cells are adhered to the surface, the top of the device could be removed and the bottom plate with the adhered cells could be stained for microscopic examination. Moreover, the bands of cells could be collected by selectively cooling to below LCST the region of the plate to which they are adhered thereby enabling the cells to be lifted off of the plate, and flowed out of the device without affecting the remaining adhered cells.

### Example 2

### General Procedure for Solution PEGylation of SiO₂-Coated Photoconductor

### Pretreatment:

Typically, a batch of ten photoconductor chips, having multilayer construction as shown in FIGS. 12-15, was pretreated prior to PEGylation. After the removal of photoresist by sonicating the chips in an organic solvent, for example, PRS-3000™ Positive Photoresist Stripper, obtained from J. T. Baker, the surfaces were rinsed thoroughly with plenty of ethanol, blow-dried with a stream of nitrogen, and baked in a convection oven at 110° C. for 30 minutes. The chips were immersed in a 20 mL-mixture of NH₄OH (29%), H₂O₂ (30%), and DI water in 1:1:4 v/v ratio and rocked on a Cole-Parmer Rocking Platform at 10 rpm for 90 minutes. The chips were removed and rinsed thoroughly with plenty of DI water, blow-dried with a steam of nitrogen, and baked in a convection oven at 110° C. for 30 minutes. The pretreated chips were used immediately for PEGylation.

### PEGylation:

A TEFLON® box constructed in such a way that it had a cavity of 35 mL and slots at the bottom to hold 10 photoconductor chips, 11 mm×20 mm in size, at vertical position, was used for solution PEGylation. The dimensions and capacity of this TEFLON® box can be scaled up to hold more chips. After placing the chips in the TEFLON® PEGylation box, the airtight cap containing an inlet and an outlet capped with rubber septums, was replaced and sealed. The PEGylation box was then purged with ultra-pure argon at 500 mL per minute for 2 minutes. A solution of 0.527 g of 2-[methoxy(polyethyleneoxy)propyl]trimethoxysilane, MW of 5,910 Da, (Nektar, Huntsville, Ala.) in 30 mL of anhydrous tetrahydrofuran (THF) was added using a syringe. It was followed by adding 1.0 mL of triethylamine. The sealed PEGylation box was rocked on a Cole-Parmer Rocking Platform at 55 rpm. After 20 hours of rocking the chips were removed, rinsed briefly with THF, blow-dried with a stream of nitrogen, and baked in a convection oven at 110° C. for 10 minutes. The PEGylated chips were kept in a covered Petri dish under ambient conditions prior to use. Static water contact angle was measured using prop Shade Analysis System DSA100 obtained from Kruss, Matthews, N.C. A total of 12 data points were taken from three random samples. The average static water contact angle was 32.1° with a standard deviation of 0.5°.

After PEGylation, electrical contact was prepared using copper tape with conductive adhesive as shown in FIG. 22.

### Example 3

### General Procedure for Solvent-Free PEGylation of SiO₂-Coated Photoconductor

### Pretreatment:

Typically, a batch of 16 photoconductor chips, having multilayer construction as shown in FIGS. 12-15, was pretreated prior to PEGylation. After the removal of photoresist by sonicating the chips in an organic solvent, for example, PRS-3000™ Positive Photoresist Stripper, obtained from J. T. Baker, the surfaces were rinsed thoroughly with plenty of ethanol, blow-dried with a stream of nitrogen, and baked in a convection oven at 110° C. for 30 minutes. The exposed ITO surface was coated with a two-parts silver conductive epoxy (EPO-TEK® E2101, Epoxy Technology, Billerica, Mass.) and cured at 50° C. overnight. The chips were soaked in 1:1:4 v/v of 29% NH₄OH, 30% H₂O₂, and deionized water at room temperature for 90 minutes. They were rinsed with deionized water thoroughly. The chips were subsequently soaked in 1:1:4 v/v mixture of HCl (38%). H₂O₂ (30%), and deionized water at room temperature for 90 minutes. The chips were rinsed with deionized water thoroughly and baked in a 110° C. convection oven for 30 minutes. The chips were optionally dipped into Piranha solution (4:1 v/v conc. H₂SO₄ and 30% H₂O₂) very briefly, rinsed with plenty of deionized water, blow-dried with a stream of nitrogen, and baked in a convection oven at 110° C. for 30 minutes. They were used immediately for PEGylation.

### Solvent-free PEGylation:

Typically, a batch of 16 chips is PEGylated in one run. The set up and process sequence for solvent-free PEGylation are shown in FIGS. 17A-C. About 10 mg of 2-[methoxy(polyethyleneoxy)propyl]trimethoxysilane, MW of 5,910 Da, (Nektar, Huntsville, Ala.) was placed on the SiO₂ surface of a chip sitting on a hot plate at 65° C. and allowed to melt. Another chip was placed onto the first chip in such a manner that the silicon dioxide surfaces were facing each other. They were allowed to sit on the hot plate for about 24 hours covered with a glass dish. The chips were removed and rinsed with plenty of water, blow-dried with a stream of nitrogen, and baked at 110° C. for 10 minutes. The PEGylated chips were kept in a covered Petri dish under ambient conditions prior to use. Static water contact angle was measured and a total of 6 data points were taken from 6 random samples. The average static water contact angle was 26.6° with standard deviation of 1.60.

### Example 4

### General Procedure for Solvent-Free PEGylation of ITO-Glass Electrodes

### Pretreatment:

Typically, a batch of eight ITO-glass electrodes was pretreated prior to PEGylation. They were rinsed thoroughly with deionized water, methanol, acetone, methanol, water, blow-dried with a steam of nitrogen, and baked in a convection oven at 110° C. for 10 minutes. The chips were then immersed in a 20 mL-mixture of NH₄OH (29%), H₂O₂ (30%), and deionized water in 1:1:4 v/v ratio and rocked on a Cole-Parmer Rocking Platform at 10 rpm for 90 minutes. The chips were removed and rinsed thoroughly with plenty of deionized water, blow-dried with a steam of nitrogen, and baked in a convection oven at 110° C. for 10 minutes. After pretreatment, an electrical contact was prepared using copper tape with conductive adhesive similar to that shown in FIG. 22. The ITO chips prepared in this manner were used immediately for PEGylation

### PEGylation:

Typically, a batch of 8 or 16 ITO-glass electrodes was PEGylated in one run. The PEGylation procedure follows that reported in Example 3. The experimental set up is illustrated in FIG. 23A-C. Static water contact angle was measured and a total of 15 data points were taken from 5 random samples. The average static water contact angle was 35.5° with a standard deviation of 2.3°.

In this application, the use of the singular includes the plural unless specifically stated otherwise. It is noted that, as used in this specification and the appended claims, the singular forms “a,” “an,” and “the,” include plural referents unless expressly and unequivocally limited to one referent. In this application, the use of “or” means “and/or” unless stated otherwise. Furthermore, the use of the term “including”, as well as other forms, such as “includes” and “included”, is not limiting. As used herein, the term “include” and its grammatical variants are intended to be non-limiting, such that recitation of items in a list is not to the exclusion of other like items that can be substituted or added to the listed items. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1.-42. (canceled)

43. A method of selectively binding a biological material to an area on a surface comprising:
coating a surface with a surface material having a lower critical solution temperature in the range of between 5° C. and 82° C.; and
adjusting the temperature of the area to change the adhesion of the biological material to the surface.

44. The method of claim 43 wherein the material is a polyacrylamide, poly(N-isopropylacrylamide), or polyacrylamide derivative.

45. The method of claim 44 wherein the polyacrylamide derivative is an N-substituted variant of acrylamide or methacrylamide

46. The method of claim 45 wherein the N-substituted acrylamide is 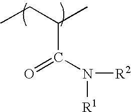
wherein R¹ is selected from hydrogen (H), methyl (Me), or ethyl (Et) and R² is selected from ethyl (Et), propyl (Pro), iso-propyl (Iso-pro), Methoxypropyl, or methyl (Me).

47. The method of claim 45 wherein the N-substituted acrylamide is 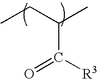
wherein R³ is selected from

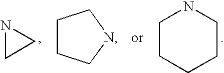

48. The method of claim 45 wherein the methacrylamide is a co-monomer.

49. The method of claim 45 wherein the methacrylamide is selected from (meth)acrylic acid, esters of (meth)acrylic acid, (meth) acrylamide, substituted (meth)acrylamides, 2-hydroxyethyl (meth)acrylate (HEMA), N-vinyl pyrrolidone (NVP), other vinyl monomers, or combinations thereof.

50. The method of claim 43 wherein adjusting the temperature comprises increasing the temperature of the material to increase adsorption of the biological material.

51. The method of claim 43 wherein adjusting the temperature comprises decreasing the temperature of the material to increase detachment of the biological material.

52. The method of claim 43 wherein the biological material is selected from biological molecules, cells, cell aggregates, organelles, stem cells, nucleic acids, bacteria, protozoans, viruses, or any combination thereof.

53. The method of claim 43 wherein the surface material includes from about 5 to about 1000 repeating units.

54. The method of claim 43 wherein the surface material is bioconjugated with a biomolecule.

55. The method of claim 43 wherein the surface material includes a carboxylic moiety.

56. The method of claim 55 wherein the carboxylic moiety is poly(ethylene glycol).

57. The method of claim 43 wherein the surface is a metal electrode.

58. The method of claim 57 wherein the metal electrode is a gold electrode.

59. The method of claim 58 wherein the gold electrode is transparent.

60. The method of claim 43 wherein the surface is selected from at least one of gold, indium tin oxide, or aluminum.

61. The method of claim 43 wherein the surface includes two electrodes.

62. The method of claim 61 wherein a power source is in electrical communication with the two electrodes.

63. The method of claim 62 wherein the power source is selected from a DC or an AC power source.

64. The method of claim 43 wherein the surface further comprises a first electrode, a second electrode, a photoconductive material, a plurality of discrete areas, and optionally a transparent layer grafted with at least one of a carboxylic moiety, an amino moiety, a poly(ethylene glycol) moiety, a polymer of (poly(ethylene oxide) methyl ether)acrylate, a poly(2-hydroxyethyl-(meth)acrylate), a poly(N-vinylpyrrolidone), a poly(N-vinylformamide), a poly(N-vinylformamide) derivative, a poly(meth)acrylamide), and a poly((meth)acrylamide) derivative.

65. A method of selecting at least a portion of a biological material in a sample comprising:
a. raising the temperature of a surface to adhere biological material located in the sample, wherein the surface is coated with a surface material having a lower critical solution temperature in the range of between 5° C. and 82° C. and
b. cooling an area of the surface to which a portion of biological material is located to release the biological material from the surface.

66. The method of claim 65 further comprising removing the released biological material from the proximity of the surface.

67. The method of claim 65 further comprising applying a stain to the biological material to identify at least one area of the surface to be cooled.

68. The method of claim 65 wherein the biological material is cells.

69. A method of sorting biological material in a sample comprising:
a. coating a surface with a material having a lower critical solution temperature in the range of between 5° C. and 82° C.;
b. introducing a sample to the coated surface;
c. sorting the biological material into areas;
d. increasing the temperature of the surface to adhere the biological material to the surface; and
e. decreasing the temperature of the areas of the surface where non-desired biological material is located.

70. The method of claim 69 wherein decreasing the temperature of the areas releases the non-desired material from the surface.

71. The method of claim 69 wherein sorting includes scanning a light source across the surface to sort the material.

72. The method of claim 71 wherein the scanning includes changing the applied field or frequency of the light source.

73. A method of selectively binding a biological material to an area on a surface comprising:
coating a surface with a surface material exhibiting thermally reversible solubility in water; and
adjusting the temperature of the area to change the adhesion of the biological material to the surface.
